(19) United States
(12) Patent Application Publication
(10) Pub. No.: US 2014/0213680 A1
(43) Pub. Date: Jul. 31, 2014

(54) POLYSULFIDE POLYOLS, THEIR PRODUCTION AND USE

(71) Applicant: BASF SE, Ludwigshafen (DE)

(72) Inventors: Marco BALBO BLOCK, Hamburg (DE); Kevin Rhudy, Woodbury, MN (US); Christoph Fleckenstein, Freigericht (DE)

(73) Assignee: BASF SE, Ludwigshafen (DE)

(21) Appl. No.: 14/166,151

(22) Filed: Jan. 28, 2014

Related U.S. Application Data
(60) Provisional application No. 61/757,761, filed on Jan. 29, 2013.

(51) Int. Cl. C08G 18/38; C08G 18/87; C08K 5/372; C08J 9/00; C07C 319/22; C07C 323/12

(52) U.S. Cl. CPC C08G 18/3863; C07C 319/22; C07C 323/12; C08K 5/372; C08J 9/00; C08G 18/87
USPC 521/174; 568/55; 524/385; 528/76; 525/539

(57) ABSTRACT

The present invention relates to polysulfide polyols, their production and use.

This invention relates to polysulfide polyols, their production and use.

There is a growing market demand for high-temperature stable polyurethane (PU) materials, in particular high-temperature stable PU rigid foams for pipe insulation. It is known to use mineral wool, which is thermally stable due to its inorganic nature, for pipe insulation. However, mineral wool has poor pipe insulation properties. On the other hand, PU-based foams show good insulation properties, but they have problems to guarantee a service life of 10 to 30 years at 190° C. and more, as the urethane bond slowly degrades at such temperatures. Isocyanurate-modified foams show improved thermal stability, but are still not sufficiently stable and possess poor flow properties in the molds. A higher cross-linking density by utilitzing higher functional polyols is not feasible due to poor flow properties in the molds. In addition, higher PIR content in the foam increases the brittleness, reducing the adhesion of the foam to the pipe and pipe cover materials. Thus, there remains a need for polyols that can be used for the production of PU-based rigid foams which are sufficiently thermally stable to be used as pipe-insulation materials. A solution for this problem could be the introduction of sulfide bonds into polyols. DD 299 187 A5 describes a process for the production of sulfur-containing organo-silicium compounds. EP 0 466 066 A1 discloses a process for production of certain oligosulfides, and U.S. Pat. No. 6,211,345 B1 describes the synthesis of cyclic sulfur silanes.

U.S. Pat. No. 3,499,863 describes the synthesis of sulfur-containing polyols. However, the process disclosed in this document starts from halogen alkanes and sodium salts for sulfur containing compounds, rather than from elemental sulfur and unsaturated alcohols as starting materials. This has the disadvantage that sodium salts (sodium halides as well as unreacted sodium precursors) have to be separated off after the reaction, leading to a tedious work-up procedure. In addition, the resulting products usually contain higher amounts of halide based impurities which is often disadvantageous.

In summary, there is no disclosure of a process for the production of sulfur-containing polyols by direct conversion of double bonds with elemental sulfur. Thus, the problem to be solved was to provide a convenient process for the production of polyols which could be used as starting materials for highly thermally stable polyurethanes.

Surprisingly, the inventors have found a process that solves the above problem.

The object of the present invention therefore is a process for the production of sulfur-containing polyols by reaction of at least one unsaturated alcohol A with elemental sulfur. In one embodiment of the inventive process, there is only one unsaturated alcohol A. In another embodiment of the inventive process, a mixture of at least two unsaturated alcohols A is used.

### Definitions

In the context of this disclosure, the term “polyol” refers to an organic compound having at least two alcohol groups per molecule. In the context of the present invention, the term “unsaturated compound” means an organic compound having at least one unsaturated carbon-carbon bond.

In a preferred embodiment of the inventive process, the sulfur-containing polyol has a viscosity of less than 10 Pa*s at 25° C., as determined according to DIN (German industry norm) ISO 2555.

The elemental sulfur may be used in solid or in liquid form.

In a preferred embodiment, the inventive process is performed neat, i. e. no solvent is used in addition to the at least one unsaturated alcohol A and the sulfur. However, it is possible to use at least one solvent. When a solvent is used, the solvent preferably has a boiling point of at least 120° C.; furthermore, the solvent preferably contains no sulfur.

Examples of solvents that may be used in the inventive process include DMF, xylene and mesitylene. Also high-boiling alcohols and ethers, for example diglyme, may be used.

It is also possible to add at least one amine compound to the reaction mixture, for example ethanolamine. However, it is preferred not to add an amine.

It is not necessary to work up the reaction mixture after the end of the reaction, for example before using the product [mixture] for the production of polyurethanes. However, the reaction mixture may also be worked up after the end of the reaction by separating off unreacted sulfur. This may be done by mechanical separation, for example by precipitation and filtration.

The inventors have shown by ¹H-NMR experiments that the reaction runs almost quantitatively, i. e. more than 99% of the unsaturated alcohols A, based on the double-bond equivalents, are reacted with sulfur.

The inventive process for the production of sulfur-containing polyols by reaction of at least one unsaturated alcohol A with elemental sulfur provides a product mixture, containing, inter alia, monosulfides, disulfides, trisulfides and tetrasulfides.

In spite of the content of sulfur, the inventive products do not smell unpleasant. On the contrary, some of the inventive products, when, for example, derived from citronellol as unsaturated alcohol A, even smell rather pleasant.

The inventive products may be used for the production of polyurethanes, in particular rigid foam polyurethanes.

Thus, a further object of the present invention is a process for the production of polyurethanes, in particular rigid foam polyurethanes, by reaction of at least one inventive sulfur-containing polyol with at least one isocyanate, optionally in the presence of a blowing agent.

Further objects of the present invention is also a process for the production of polyurethanes, in particular rigid foam polyurethanes, by reaction of at least one inventive sulfur-containing polyol with at least one unsaturated polyol and with at least one isocyanate, optionally in the presence of a blowing agent, and a process for the production of polyurethanes, in particular rigid foam polyurethanes, by reaction of at least one inventive sulfur-containing polyol with at least one unsaturated polyol and with at least one isocyanate, in the presence of a vulcanization catalyst and optionally in the presence of a blowing agent. Typical vulcanization catalysts, also referred to as vulcanization accelerators, are benzothiazols, thiuramdisulfides, dithiocarbamates, guanidines, thioureas. They are added to the polyol blend in amounts between 0.25 and 5 w % based on the total weight of the polyol blend. Typical vulcanization activators are Zinc salts of fatty acids or blends of Zinc oxide and fatty acids. They are added to the polyol blend in amounts between 0.25 and 5 w % based on the total weight of the polyol blend. An overview about accelerators and activators and their catalyzing mechanism as well as the reactions occuring during the polysulfide polyol synthesis as well as during the post-curing in the PU product is given in: Kautschuktechnologie, F. Rothemeyer and F. Sommer, Hanser Verlag, 2001, München, Wien and the references therein. Further vulcanization accelators which can be used, alone or in combination, in the present invention include:
Thiazoles, for example:
2-Mercaptobenzothiazole (CAS #: 149-30-4)
   Dibenzothiazole disulfide (CAS #: 120-78-5)
   2-Mercaptobenzothiazole Zinc salt (CAS #: 155-04-4)
Sulphenamides, for example:
   N-Cyclohexyl-2-benzothiazole sulfenamide (CAS #: 95-33-0)
   N-Oxydienthylene-2-benzothiazole sulfenamide (CAS #: 102-77-2)
   N-tert-butyl-2-benzothiazyl sulfenamide (CAS #: 95-31-8)
Guanidines, for example:
   Diphenyl guanidine (CAS #: 102-06-7)
   Di-o-tolylguanidine (CAS #: 97-39-2)
Thiurams, for example:
   Tetramethyl thiuram disulfide (CAS #: 137-26-8)
   Tetraethyl thiuram disulfide (CAS #: 97-77-8)
   Tetramethyl thiuram monosulfide (CAS #: 97-74-5)
   Isobutyl thiuram disulfide (CAS #: 3064-73-1)
   Tetrabenzylthiuram disulfide (CAS #: 10591-85-2)
   Dipentamethylene thiuramtetrasulfide (CAS #: 120-54-7)
Dithiocarbamates, for example:
   Zinc dimethyl dithiocarbamate (CAS #: 137-30-4)
   Zinc diethyl dithiocarbamate (CAS #: 14324-55-1)
   Zinc dibutyl dithiocarbamate (CAS #: 136-23-2)
   Zinc N-ethyl-dithiocarbamate (CAS #: 14634-93-6)
   Zinc dibenzyl dithiocarbamate (CAS #: 14726-36-4)
   Copper dimethyl dithiocarbamate (CAS #: 137-29-1)
Thioureas, for example:
   Ethylene thiourea (CAS #: 96-45-7)
   N,N′-Diethylthiourea (CAS #: 105-55-5)
   N-N′-Diphenylthiourea (CAS #: 102-08-9)

In a preferred embodiment of the inventive process for the production of polyurethanes, an additional step of post-curing is performed.

“Post-curing”, in the context of this disclosure, refers to a prolonged thermal exposure to ensure a certain amount of reaction between the polysulfide polyol and the unsaturated polyol within the rigid foam. If the post-curing is performed before the application then post-curing is defined as storage of the PU at ambient humidity or dry conditions for 30min to 14 days (preferably 1-3 days) at 130-220° C. (preferably 160-190° C.). The PU can also be submitted to more than one post-curing treatment, if desired. The post-curing leads presumably to an additional crosslinking in the PU. The improvement of mechanical properties of the PU vary with varying post-curing conditions.

Thus, the inventive products can be used as high-temperature stable PU rigid foams, in particular for pipe insulation.

### EXAMPLES

Sulfur-containing polyols were synthesized as follows.

The OH number was determined in accordance with DIN 53240 (DIN=“deutsche Industrienorm”, German industry norm).

### Example 1

### Reaction of Oleic Alcohol with elemental Sulfur

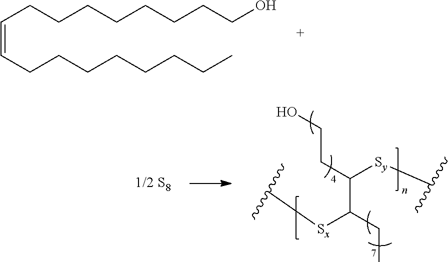

In a 250 mL standard glass stirring apparatus, elemental sulfur (20.5 g, 0.08 mol) and oleic alcohol (43.0 g, 0.16 mol) are dissolved in 50 mL DMF. Then the suspension is heated to 140° C.

At 120° C. the reaction mixture became a homogeneous solution.

After 16 hours quantitative conversion of the alcohol was shown (reaction control via ¹H-NMR).

The reaction mixture was cooled down to ambient temperature, then 200 mL methylene chloride and 200 mL water were added. The mixture was stirred for 15 min, then 2 spoons of celite were added and the mixture was filtered via suction filtration (glass fritt, (Por. 3)). The filtrate was transferred into a separation funnel, the lower organic phase was separated and all volatiles were removed via a rotary evaporator (60° C. bath temperature, 1 mbar) to afford a viscous dark reddish oil with small crystals. In order to remove non reacted sulfur from the mixture, the oil was dissolved in 150 mL MTBE and filtered over celite. The filtrate was collected and all volatiles were removed (70° C., 0.1 mbar) to afford the product as a dark reddish oil (59 g).

Analytical Data:

Elemental Analysis: found: C 56.3%, O 4.6%, H 9.5%, S 29.1%.

OH-Number: 134 mg KOH/g

### Example 2

### Reaction of Citronellol with elemental Sulfur

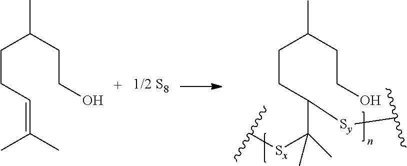

In a 500 mL standard glass stirring apparatus, elemental sulfur (82.1 g, 0.32 mol) was suspended in citronellol (100 g, 0.59 mol). Then the suspension was stirred at 140° C.

After 18 hours quantitative conversion of the alcohol was shown (reaction control via ¹H-NMR).

The reaction mixture was cooled down to ambient temperature.

In order to remove non reacted sulfur from the mixture, the oil was dissolved in 450 mL MTBE and filtered over celite (3 spoons). The filtrate was collected and all volatiles were removed (70° C., 0,1 mbar) to afford the product as a dark reddish, viscous oil (152 g).

Analytical Data:

Elemental Analysis: found: C 46.2%, O 6.6%, H 7.7%, S 40.2%.

OH-Number: 177 mg KOH/g

### Example 3

### Reaction of 10-Undecen-1-ol with Elemental Sulfur

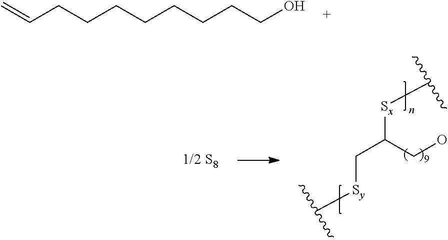

In a 500 mL standard glass stirring apparatus, elemental sulfur (74.4 g, 0.29 mol) was suspended in citronellol (100 g, 0.59 mol). Then the suspension was stirred at 140° C.

After 11 hours quantitative conversion of the alcohol was shown (reaction control via ¹H-NMR).

The reaction mixture was cooled down to ambient temperature.

In order to remove non reacted sulfur from the mixture, the oil was dissolved in 450 mL MTBE and filtered over celite (3 spoons). The filter cake was washed with THF (3×100 mL). The filtrates were combined and all volatiles were removed (70° C., 0.1 mbar) to afford the product as a dark brown, viscous oil (61 g).

Analytical Data:

Elemental Analysis: found: C 50.1%, O 6.8%, H 8.5%, S 35%.

Furthermore, foaming tests were done with mixtures containing sulfur-containing polyols, as well as with mixtures not containing sulfur-containing polyols.

All compression strengths are average values (standardized to density, determined geometrically) from three measurements. (Alle Druckfestigkeiten sind dichte-normierte, geometrisch gemittelte Mittelwerte aus drei Messungen.)

Substances that were used:

Krasol LBH-P 2000: polybutadiene diol, fn=1.9, OH no.=51

Tetraethylene glycol: fn=2, OH no.=577

Polythio-dipropyl-diol: fn=2, OH no.=595

Sovermol 1102: biobased ether-ester polyol, fn=2.1, OH no.=227

Polysulfide from Undecenole: fn=2, OH no.=238

Zinc stearate: vulcanization activator

Tetramethyl thiuram disulfide: vulcanization catalyst/-accelerator

Results: table 1

| | Comp. 1 | Exp. 1 | Comp. 2 | Exp. 2 |
|---|---|---|---|---|
| Other polyols | 78 | 78 | 78 | 78 |
| Krasol LBH-P 2000 | 10 | 10 | 10 | 10 |
| Zinc stearate | 1 | 1 | 1 | 1 |
| Tetramethyl-thiuram-disulfide | 1 | 1 | 1 | 1 |
| Tetraethylenglykol | 10 | | | |
| Polythio-dipropyl-diol | | 10 | | |
| Sovermol 1102 | | | 10 | |
| Polysulfide from undecenol | | | | 10 |
| mixture of catalysts | 2 | 2 | 2 | 2 |
| Water | 1.5 | 1.5 | 1.5 | 1.5 |
| Cyclopentane, 95% | 6.5 | 7.5 | 7 | 6.5 |
| PMDI | 253 | 254 | 228 | 228 |
| NCO-index | 310 | 310 | 310 | 310 |
| free-rise-density (g/l) | 54.6 | 54.8 | 54.3 | 54.3 |
| Foamed to cubes in a closed | | | | |
| form with Überpackungs- | | | | |
| faktor 1.15 | | | | |
| Compression strength of fresh | 0.31 | 0.32 | 0.30 | 0.29 |
| foam, N/mm² | | | | |
| Compression strength after | 0.28 | 0.36 | 0.30 | 0.32 |
| thermal stress, 48 h at | | | | |
| 190° C., N/mm² | | | | |
| growth after tempering, | −0.03 | 0.04 | 0.00 | 0.03 |
| N/mm²: | | | | |
| pressure-E-module fresh | 9.00 | 8.00 | 7.60 | 7.70 |
| foam, N/mm² | | | | |
| Pressure-E-module after | 9.00 | 8.70 | 8.00 | 8.50 |
| thermal stress, 48 h at | | | | |
| 190° C., N/mm² | | | | |
| growth after tempering, | | | | |
| N/mm²: | | | | |

1. A process for producing a sulfur-comprising polyol, comprising reacting, in a reaction mixture, an unsaturated alcohol with elemental sulfur, wherein the unsaturated alcohol has only one carbon-carbon-double bond-unsaturation per molecule.

2. The process of claim 1, wherein the unsaturated alcohol has 1-4 OH-groups.

3. The process of claim 1, wherein the unsaturated alcohol has a molecular weight of less than 300 g/mol.

4. The process of claim 1, wherein at least one unsaturated alcohol is selected from the group consisting of oleyl alcohol, citronellol, 10-undecen-l-ol, linaool, geraniol, farnesol, and cinnamyl alcohol.

5. The process of claim 1, wherein a temperature during the reaction from 100 to 200° C..

6. The process of claim 1, wherein the reaction takes between 0.5 and 20 hours.

7. The process of claim 1, wherein a molar ratio of sulfur to double bond equivalents of unsaturated alcohol is from 1 to 9 mole sulfur per double bond equivalent.

8. The process of claim 1, wherein no solvent is used employed.

9. The process of claim 1, wherein the reacting is performed in a solvent, the solvent has a boiling point of at least 12020 C. and the solvent does not comprise sulfur.

10. The process of claim 1, further comprising adding an amine compound to the reaction mixture.

11. The process of claim 1, further comprising adding at least one compound selected from the group consisting of thiazoles, sulphenamides, guanidines, thiurams, dithiocarbamates and thioureas to the reaction mixture.

12. The process of claim 1, wherein the reaction mixture does not comprise an amine compound.

13. The process of claim 1, further comprising working up the reaction mixture after an end of the reaction by separating off unreacted sulfur.

14. The process of claim 1, wherein the reaction mixture is not worked up after the end of the reaction.

15. A sulfur-comprising polyol, obtained by the process of claim 1.

16. A polymer composition, comprising a polymer X and the sulfur-comprising polyol of claim 15.

17. A process for producing a polyurethane, comprising reacting the sulfur-comprising polyol of claim 15 with an isocyanate, optionally in the presence of a blowing agent.

18. A process for producing a polyurethane, comprising reacting the sulfur-comprising polyol of claim 15 with an unsaturated polyol and with an isocyanate, optionally in the presence of a blowing agent.

19. The process of claim 18, wherein the unsaturated polyol is a polybutadiene diol.

20. The process of claim 17, comprising reacting the sulfur-comprising polyol with an unsaturated polyol and with the isocyanate, in the presence of a vulcanization catalyst and optionally in the presence of a blowing agent.

21. The process of claim 17, further comprising performing post-curing.

22. The process of claim 17, further comprising performing in-situ post-curing during a high-temperature resistance test or during a product application time.

23. The process of claim 17, wherein the polyurethane is a rigid foam polyurethane comprising an isocyanurate and having a NCO-index>200.

24. A rigid foam polyurethane obtained by the process of claim 17, wherein the rigid foam polyurethane is suitable for insulating a pipe.
